(19) United States
(12) Patent Application Publication
(10) Pub. No.: US 2010/0298772 A1
(43) Pub. Date: Nov. 25, 2010

(54) TRANCUTANEOUS DEVICES AND KITS THAT PROVIDE CUES FOR LOCATION OF INSERTION SITE, EXIT SITE AND DEVICE PATH, AND METHODS OF USE

(76) Inventors: Matthew John Moore, Ephrata, PA (US); Joel Rosenblatt, Pottstown, PA (US); Daniel J. Spangler, Forth Worth, TX (US); Hiep Do, Sinking Spring, PA (US); Onajite Okoh, Reading, PA (US)

Correspondence Address: AMSTER, ROTHSTEIN & EBENSTEIN LLP, 90 PARK AVENUE, NEW YORK, NY, 10016, US

(21) Appl. No.: 12/733,997

(22) PCT Filed: Oct. 2, 2008
(86) PCT No.: PCT/US2008/011388

§ 371 Date: Jun. 15, 2010

Related U.S. Application Data
(60) Provisional application No. 60/997,400, filed on Oct. 3, 2007.

(51) Int. Cl. A61M 25/01; A61B 17/3209; A61B 17/34

(52) U.S. Cl. 604/116; 606/167; 606/185

(57) ABSTRACT

Transcutaneous devices are disclosed that self-provide cues for exact location of transcutaneous insertion sites, exit sites, and/or the track of the device between the sites, as are methods and kits for using the transcutaenous devices to mark the sites and track of the device and for removing location cues.

### CROSS-REFERENCE TO RELATED APPLICATION

This application claims the benefit of U.S. Provisional Patent Application No. 60/997,400, filed on Oct. 3, 2007, the content of which is hereby incorporated by reference into the subject application.

### FIELD OF THE INVENTION

The present invention relates to transcutaneous devices, such as catheters, that self-provide cues for exact location of insertion and exit sites and the path of the device in a subject, to methods of using the devices to mark the sites, to methods of removing cues provided by the devices, and to kits including the devices and materials for removing location cues.

### BACKGROUND OF THE INVENTION

Various publications are referred to throughout this application. Full citations for these references may be found at the end of the specification immediately preceding the claims. The disclosures of these publications are hereby incorporated by reference in their entireties into the subject application to more fully describe the art to which the subject application pertains.

Transcutaneous devices are routinely used in clinical settings. Examples of such transcutaneous devices include sutures, staples, trocars, fixation pins or clips, wires or cables, and catheters. Providing a detectable marking of the insertion and exit sites of transcutaneous devices can be an important aid for clinicians following removal of the devices. In some cases the exact location of the site might be detectable from contrast provided by the puncture wound, but in other cases (particularly small devices) it may be difficult to locate the site a short time after removal of the device.

A clear indicator of where a transcutaneous device was inserted can assist in providing targeted site care to patients with impaired wound healing following device removal. In cases where a catheter is removed it may be a benefit for certain patients to know where the previous catheter had been inserted in order to avoid using the same site or vein. This can help reduce the risk of infection as well as reduce the likelihood of vascular complications such as phlebitis or deep vein thrombosis by avoiding repeated mechanical trauma to the same vein. For other patients who have veins of poor quality, bleeding disorders or veins that are difficult to insert into, the risks of a failed insertion procedure may render it beneficial to reuse the same insertion track.

Various methods and devices have been used to identify the location of body sites. For example, surgical marking pens have been used to indicate the location of device insertion (e.g., TLS™ surgical marker by Porex Surgical Products Group or Convertors® Surgical Marking Pens by Cardinal Health). Also described are syringes that dispense a colorant (U.S. Pat. No. 5,192,270), devices that impress a mark into the skin (U.S. Pat. No. 5,147,307), tissue-marking probes (U.S. Pat. No. 6,780,179), and biodegradable polymers for marking tissue and sealant tracts (PCT International Publication No. WO 03/093338 A2). Implantable medical markers have been described that designate location but require implantation of a marker device (U.S. Pat. No. 6,228,055; PCT International Publication No. WO 2006/022786). U.S. Pat. No. 5,713,858 describes a method of repeatedly accessing body tissue using a permanently implantable guiding catheter, which does not involve a cue for the site of removal. Systems and methods for marking body cavities have also been described (U.S. Patent Application Publication No. 2006/0200024). Numerous publications teach the application of antimicrobial dyes to medical devices (e.g., U.S. Pat. Nos. 5,607,417, 5,709,672, 6,361,786, 6,551,346, 6,617,142; U.S. Patent Application Publications 2003/0078242 A1, 2005/0131356 A1 and 2005/0197634 A1). However, a device such as a catheter that self-marks its transcutaenous site upon insertion and/or removal has not been reported.

### SUMMARY OF THE INVENTION

The present invention provides transcutaneous devices for marking an insertion site and/or an exit site of the device through the skin of a subject and/or the track of the device through the subject, where the transcutaneous device provides a cue in an amount effective to mark the insertion site, the exit site and/or the track of the transcutaneous device.

The present invention also provides methods of marking an insertion site and/or an exit site of the transcutaneous device and/or the track of the device through the subject, where the method comprises inserting the transcutaneous device through the skin of the subject, where the transcutaneous device provides a cue in an amount effective to mark the insertion site, the exit site, and/or the track of the transcutaneous device.

The invention further provides methods of decoloring oxidizable location cue dye stains, where the method comprises applying peroxide to the stain and treating the peroxide with non-ionized metal particles.

The invention provides kits for removing location cue dye stains, where the kit comprises peroxide and non-ionized metal particles.

The invention also provides transcutaneous device kits for marking an insertion site and/or an exit site of the transcutaenous device on the dermis and/or marking the track of the transcutaneous device, and for decoloring the dermal mark and/or decoloring the mark along the track of the transcutaneous device, where the kit comprises a transcutaneous device that provides an oxidizable visible dye mark on the surface of the skin to mark the insertion site and/or the exit site and/or a dye mark along the track of the transcutaneous device; and non-ionized metal particles, and optionally peroxide, for use in removal of the mark.

### BRIEF DESCRIPTION OF THE DRAWINGS

FIG. 1. HPLC chromatogram of 50 μg/mL gentian violet (GV) (ScienceLab)/200 μg/mL CHA standard in water/ACN/MeOH @ 588 nm.

FIG. 2. HPLC chromatogram of gentian violet compounded in Tecothane® 2095A @ 588 nm.

FIG. 3. HPLC chromatogram of gentian violet Standard (Aldrich) @ 588 nm.

FIG. 4. HPLC chromatogram of gentian violet from Tecothane® 2095A Tube sample @ 588 nm.

FIG. 5. Chromatogram at 588 nm of Tecothane® (w/30% BiOCl) compounded with 0.5% GV (Aldrich ACS grade) at 170° C.

FIG. 6. Chromatogram at 588 nm of degraded Tecothane® (w/30% BiOCl) compounded with 0.5% GV (Aldrich ACS grade) at 217° C.

FIG. 7. Chromatogram at 253 nm of Tecothane® (w/30% BiOCl) compounded with 0.5% GV (Aldrich ACS grade) at 170° C.

FIG. 8. Chromatogram at 253 nm of degraded Tecothane® (w/30% BiOCl) compounded with 0.5% GV (Aldrich ACS grade) at 217° C.

FIG. 9. Chromatogram at 280 nm of Tecothane® (w/30% BiOCl) compounded with 0.5% GV (Aldrich ACS grade) at 170° C. 5.896 min peak is due to solvent.

FIG. 10. Chromatogram at 280 nm of degraded Tecothane® (w/30% BiOCl) compounded with 0.5% GV (Aldrich ACS grade) at 217° C. 5.974 min peak is due to solvent.

FIG. 11. Pig skin staining setup for marking sites of transcutaneous catheter impregnation. Twenty-two different catheter treatments representing different ratios and concentrations of gentian violet and chlorhexidine were evaluated.

FIG. 12. Illustration of pig skin staining results from setup of FIG. 11.

FIG. 13A-13B. Removal of topical gentian violet (GV) stain on plated agar by silver activated hydrogen peroxide. A) 10 μl 0.1% GV+100 μl H₂O₂; B) 10 μl 0.1% GV+100 μl H₂O₂+0.00867 g Ag.

### DETAILED DESCRIPTION OF THE INVENTION

The invention provides a method of marking an insertion site, an exit site and/or the track of a transcutaneous device, the method comprising inserting a transcutaneous device through the skin of the subject, where the transcutaneous device provides a cue in an amount effective to mark the insertion site, the exit site, and/or the track of the transcutaneous device.

The invention also provides a transcutaneous device for marking an insertion site and/or an exit site of the device through the skin of a subject and/or for marking the track of the device through the subject, where the transcutaneous device comprises a cue in an amount effective to mark the insertion site, the exit site and/or the track of the transcutaneous device.

The transcutaneous device can provide a cue that marks the insertion site of the transcutaneous device, the exit site of the transcutaneous device, both the insertion site and the exit site of the transcutaneous device and/or the track of the device within the body. The insertion site and the exit site can be the same site or different sites. The device can mark the track of the device through tissues, vessels and cavities of a subject by marking surfaces the device contacts inside the body or within body cavities, such as, for example, the gastrointestinal system, auditory canals, respiratory system, nasal canals, and urinary system.

The transcutaneous device can provide a cue on the surface of the skin to mark the insertion site and/or the exit site. The cue on the surface of the skin can delineate the circumference of the portion of the transcutaneous device that is inserted through the skin and where it transited the skin. For example, a cue can be a circle on the epidermis providing this delineation. The transcutaneous device can provide a cue to mark all or part of the path of the device through tissue, vessel and cavities between the insertion site and the exit site. A cue can mark all or part of the tunnel track of the device through both hard and soft tissues.

Selective marking can be accomplished, for example, by applying the cue to only desired regions of the transcutaneous device and also applying a water soluble mask coating to those regions on top of the cue. The soluble mask coating layer would need to dissolve first before the cue could transfer. This would also prevent the cue from transferring to the implanter during the implantation procedure. Examples of water soluble coatings include, but are not limited to, carbohydrates (such as sugars or dextrans), starches, polymers (polyvinyl pyrrolidones, polyethylene oxide, polyvinyl alcohols), proteins (albumin, gelatin), biopolymers (carboxymethylcellulose, alginates, chitosans) and salts. A similar effect can be accomplished using a surface coating that hydrolyzes rather than dissolves (such as polyester materials like poly glycolic acid, poly lactic acid, polycaprolactone, polydioxanone and copolymers). A coating can be removed by enzymatic degradation rather than hydrolysis or dissolution (lipids, collagen or other insoluble proteins). For a device that partially hydrates, the cue can also be embedded in a layer below the surface of the device which requires time following the insertion to hydrate and diffuse to the surface. Only the regions of the device to which the cue is embedded would transfer the cue.

Cues include visible dyes. Preferably, the skin is marked with a color that provides a clear visual contrast to the recipient's skin color. The cue can be an agent that is detected only when an excitation wavelength of electromagnetic energy is applied to the skin. The agent can be, for example, but not limited to, a fluorescent dye, a phosphorescent dye or an x-ray agent. Examples of fluorescent dyes include, for example, coumarins, napthalimides, perylenes, rhodamines, benzanthrones, benzoxanthones and benzothioxanthones. Examples of phosphorescent dyes include, for example, zinc, calcium and strontium sulfides or aluminates with activation agents such as copper, bismuth or manganese. Examples of x-ray agents include, for example, barium, bismuth and other metal oxides and their salts. The excitation can be in the visible range of the electromagnetic spectrum but may also be outside of the visible range. The cue can be a dye that is detected outside the visible electromagnetic range. Examples of such dyes include, but are not limited to, ultra violet (UV) or blacklight visible dyes, such as pteridines, or infra red (IR) visible dyes, such as squaraines. Dyes that emit outside of the visible range can be used with special viewing equipment that converts the emission wavelengths to a visible signal or detects them in another way, for example using an electrical signal. The cue can be provided by acoustic contrast, thermal contrast or tactile contrast. For example, cues could be microspheres/microparticles that provide surface roughness or if hollow or contain bubbles could be detected by ultrasound. Thermochromic dyes (such as fluoran dyes) can be developed by local heating. The cue can be invisible unless a second component (an indicator component) is applied to the skin and then can be imaged or otherwise detected in the region where the indicator and cue overlap. Examples of such cues and indicator components include, but are not limited to: phenolpthalein ink developed by a base (such as sodium carbonate); thymolpthalein similarly developed; a base developed by ninhydrin; leuco dyes (including oxazines, Leuco Crystal Violet, tris(4-diethylamino o-tolyl)methane, bis(4-diethylamino-o-tolyl)phenylmethane, bis(4-diethylamino-o-tolyl)-thienyl-2-methane, bis(2-chloro-4-diethylaminophenyl)phenylmethane, 2-(2-chlorophenyl)amino-6-N,N-dibutylamino-9-(2-methoxycarbonyl)phenylxant hene, 2-N,N-dibenzylamino-6 N,N-diethylamino9-(2-methoxycarbonyl)phenylxanthene, benzo[a]-6-N,N diethylamino 9-(2-methoxycarbonyl)phenylxanthene, 2-(2-chlorophenyl) amino-6-N,N-dibutylamino-9-(2-methylphenylcarboxyamido)phenylxanthene, 3,6-dimethoxy-9-(2-methoxycarbonyl) phenylxanthene, 3,6-diethoxyethyl-9 (2-methoxycarbonyl)phenylxanthene, benzoyl leuco Methylene Blue and 3,7-bisdiethylaminophenoxazine) developed by oxidizing agents (such as azides or organic halogens); starch developed by iodine; iron sulfate developed by sodium sulfide; iron sulfate developed by sodium carbonate; and copper sulfate developed by sodium carbonate.

The transcutaneous device can be coated with the cue and/or impregnated with the cue. The cue can be transmitted directly from the transcutaneous device to the skin through contact. The cue can be applied to the transcutaneous device and then can rub off or slowly leach by diffusion from the transcutaneous device into the adjacent tissue. In this case it may be desirable to only apply enough cue so as to mark a small annular region of skin around the insertion site directly abutting the transcutaneous device. The degree of marking can be controlled by the loading concentration of the cue on the device.

The cue can be, for example, a triarylmethane dye, a monoazo dye, a diazo dye, an indigoid dye, a xanthene dye, a fluorescein dye, an anthraquinone dye, a quinoline dye, or mixtures thereof. The cue can be, for example, gentian violet, crystal violet, ethyl violet, methyl violet, brilliant green, methylene blue, toluidine blue, methylene violet, azure A, azure B, azure C, brilliant cresol blue, thionin, methylene green, bromcresol green, gentian acridine orange, brilliant green, acridine yellow, quinacrine, trypan blue, trypan red, gendine, genlenol, genlosan, genfoctol, or mixtures thereof.

A preferred cue is gentian violet. Gentian violet can be present in the portion of the transcutaenous device that passes through the skin in a concentration greater than 1% by weight of the portion of the device that passes through the skin. In different examples, the concentration of gentian violet in the device is greater than 1.1%, 1.5%, 2%, 2.5%, 3%, 4%, 5%, 7.5% or 10% by weight. The concentration of gentian violet can be, for example, up to 20% by weight. Gentian violet can be present in the portion of the transcutaenous device that passes through the skin in a concentration of at least 15 μg gentian violet per cm² area of the device and preferably in a concentration of 50-500 μg/cm².

The transcutaneous device can include a single type of cue, or a combination of two or more types of cues.

The transcutaneous device can be, for example, a suture, a staple, a trocar, a tube, a multilumen tube, multiple tubes, a fixation pin, a fixation clip, a wire, a cable or a catheter. A preferred transcutaneous device is a catheter.

Transcutaenous devices such as catheters can be made, for example, from one or more of silicone elastomers, thermoplastics, polyurethanes, fluoropolymers, and polyolephins. Methods of processing dyes such as gentian violet can include, for example, extrusion, injection molding, blow molding, compression molding, or any other hot melt processes. Prior to extrusion, the polyurethane resin can be precoated with the dye or the polyurethane resin can be compounded with dye powder. Devices such as catheters can also be prepared, for example, by a method comprising extruding gentian violet with a thermoplastic such as polyurethane at a temperature below 207° C., where the concentration of gentian violet is 0.05% to 20% by weight of the thermoplastic. The thermoplastic used in this method is preferably a polyurethane having a low processing temperature (below 207° C.), such as for example one or more of Tecothane®, Carbothane®, Tecoflex®, Tecophilic®, Texin® and Pellethane®. Polyurethanes or other thermoplastics that require processing temperatures above the temperature at which degradation of dye occurs are not suitable for this method unless the thermoplastic is treated, e.g. by plasticization, to lower its processing temperature below the temperature at which the dye degrades.

Also provided are cue-containing transcutaneous devices prepared by any of the methods disclosed herein. One example of such a transcutaneous device is a catheter comprising a thermoplastic such as a polyurethane having a processing temperature below 207° C., where the concentration of gentian violet in at least the transcutaneous portion of the device is 0.05% to 20% by weight of the thermoplastic.

It may be desirable for the cue to remain detectible for some time following removal of the inserted transcutaneous device but not be permanent (e.g., eventually be cleared for example by wearing or cleaning off).

The invention provides a method of decoloring a dye stain on the surface of the dermis and/or along the track of a transcutaneous device, the method comprising applying peroxide to the stain and treating the peroxide with non-ionized metal particles. Examples of non-ionized metals include, but are not limited to, silver, gold, platinum, copper, iron and zinc. Silver is a preferred non-ionized metal. Nanoparticles are a preferred type of particle. Silver nanoparticles are most preferred. Examples of peroxides include, but are not limited to, hydrogen peroxide, inorganic peroxides (magnesium, calcium, and barium peroxide), peroxyacids, peresters, benzoyl peroxide, acetone peroxide, and methyl ethyl ketone peroxide. Hydrogen peroxide is a preferred peroxide. Preferably, the dye stain is a gentian violet dermal stain. Preferably, the molar ratio of gentian violet:silver is less than 1:1.

The invention further provides a kit for removing a dye stain on the surface of the dermis and/or along the track of a transcutaneous device, the kit comprising peroxide and non-ionized metal particles. Examples of non-ionized metals include, but are not limited to, one or more of silver, gold, platinum, copper, iron and zinc. Silver is a preferred non-ionized metal. Nanoparticles are a preferred type of particle. Silver nanoparticles are most preferred. Examples of peroxides include, but are not limited to, hydrogen peroxide, inorganic peroxides (magnesium, calcium, and barium peroxide), peroxyacids, peresters, benzoyl peroxide, acetone peroxide, and methyl ethyl ketone peroxide. Hydrogen peroxide is a preferred peroxide. Kits with an inorganic peroxide, such as magnesium, calcium, or barium peroxide, may also comprise an acid such as sodium bisulfate.

The invention also provides a transcutaneous device kit for marking an insertion site and/or an exit site of the transcutaenous device on the dermis and/or marking the track of the transcutaneous device, and for decoloring a dye stain on the surface of the dermis and/or along the track of the transcutaneous device, the kit comprising: a) a transcutaneous device that provides a visible dye mark on the surface of the skin to mark the insertion site and/or the exit site and/or a dye mark along the track of the transcutaneous device, and b) non-ionized metal particles. Examples of non-ionized metals include, but are not limited to, silver, gold, platinum, copper, iron and zinc. Silver is a preferred non-ionized metal. Nanoparticles are a preferred type of particle. Silver nanoparticles are most preferred. The kit can further comprise a peroxide, such as for example, hydrogen peroxide, inorganic peroxides (magnesium, calcium, and barium peroxide), peroxyacids, peresters, benzoyl peroxide, acetone peroxide, and methyl ethyl ketone peroxide. Hydrogen peroxide is a preferred peroxide. Kits with an inorganic peroxide, such as magnesium, calcium, or barium peroxide, may also comprise an acid such as sodium bisulfate. Preferably, the transcutaneous device provides a gentian violet dye mark on the skin. The device can also mark all or part of the track of the device through the subject.

The present invention is illustrated in the following Experimental Details section, which is set forth to aid in the understanding of the invention, and should not be construed to limit in any way the scope of the invention as defined in the claims that follow thereafter.

### Experimental Details

### Examples I-II

### Incorporation of Gentian Violet into Tecothane®-2095A Resin

Experiments were performed to incorporate gentian violet into Tecothane®-2095A resin by compounding and extrusion processes. In these examples gentian violet was coated on Tecothane®-2095A pellets by soaking the resin in gentian violet/ethanol mixture and the solvent was evaporated off at ambient conditions. The gentian violet coated pellets were then fed into an extruder or compounder to making tubing or strand pellitized pellets. The gentian violet could also have been fed as a powder directly with the polymer resin for compounding and extrusion. Surprisingly, much higher loadings of gentian violet could be achieved using the high temperature process disclosed herein than had been previously disclosed without degradation of the chemical structure of the gentian violet.

### Example 1

### Compound Tecothane®-2095A Resin with 0.5% Gentian Violet

5 g of gentian violet (Sciencelab, Houston, Tex.) was dissolved in 250 ml 99% ethanol (Sigma-Aldrich, St. Louis, Mo.). 1000 g of Tecothane® 2095A (Noveon, Cleveland, Ohio) resin was added in to the gentian violet/ethanol solution. The ethanol solvent was evaporated off in a chemical fume hood overnight at ambient conditions. The gentian violet coated pellets were then dried at 50° C. and 30 inches Hg for 24 hrs prior to compounding.

The dried gentian violet coated resins were starve-fed into a 18 mm Leistritz intermeshing twin screw extruder (Somerville, N.J.) from a K-tron feeder (Pitman, N.J.) at a rate of 2.5 kg/hr. The extruder was set at 231 rpm for screw speed and the barrel zone temperatures were set from 329° F. (165° C.) thru 338° F. (170° C.). The extrudate was pelletized into small pellets.

### Example 2

### 2% Gentian Violet Loaded Tecothane® Tube

20 g of gentian violet (Sigma-Aldrich, St. Louis, Mo.) was dissolved in 1000 ml ethanol (Sigma-Aldrich, St. Louis, Mo.). 1000 g of Tecothane® resin was added into the gentian violet/ethanol solution. The ethanol solvent was evaporated off in the chemical fume hood overnight at ambient conditions. The gentian violet coated resin then dried at 65° C. and 30 inches Hg for 4 hrs prior to compounding.

The dried gentian violet coated resins were gravity fed into a ⅝′ Randcastle single screw (Cedar Grove, N.J.) microextruder. The microextruder was set at 20 rpm for screw speed and barrel zone temperatures were set from 360° F. thru 375° F. A 5 Fr tubing was drawn from a BH25 tooling (San Marcos, Calif.).

### Characterization of Gentian Violet Compounded in Tecothane® via HPLC

Gentian violet contents from compounded resin and tube sample were analyzed via HPLC method. HPLC analysis on GV loaded resin or compounded pellets was performed by weighing 0.1-0.3 g, which is roughly equivalent to 10-20 pellets (depending on the weight of the raw material used) of each formulation (n=3) and digesting in THF (5 mL or 7.5 mL) in 50 mL centrifuge tubes. Samples were allowed to sit for 45 minutes, and then vortexed until all of the polymer dissolved. An equal amount of deionized (DI) water was then added (5 or 7.5 mL), and the samples were again vortexed for 5 minutes, and then centrifuged for 10 minutes. A portion of each sample was then added to an HPLC vial and capped prior to HPLC analysis.

HPLC analysis on tubing was performed by cutting and measuring 1 cm segments of each formulation (n=3) and digesting in THF (10 mL or 20 mL) in 50 mL centrifuge tubes. Samples were allowed to sit for 45 minutes, and then vortexed for 5 minutes, or until none of the polymer was stuck to the bottom of the tubes. An equal amount of deionized (DI) water was then added (10 or 20 mL), and the samples were again vortexed for 5 minutes, and then centrifuged for 10 minutes. A portion of each sample was then added to an HPLC vial and capped prior to HPLC analysis.

HPLC analysis was performed on an Agilent 1200 Series LC using an Agilent Eclipse XDB-CN 5μ 4.6×150 mm column with the corresponding guard column. A gradient program was run using two solvent reservoirs:

MP A: 100% DI Water/0.2% Trifluoroacetic Acid,

MP B: 100% Acetonitrile/0.2% Trifluoroacetic Acid.

### Program: 0-5 min 35% B, 5-6 min 35-40% B, 6-12 min 40% B, 12-16 min 35% B.

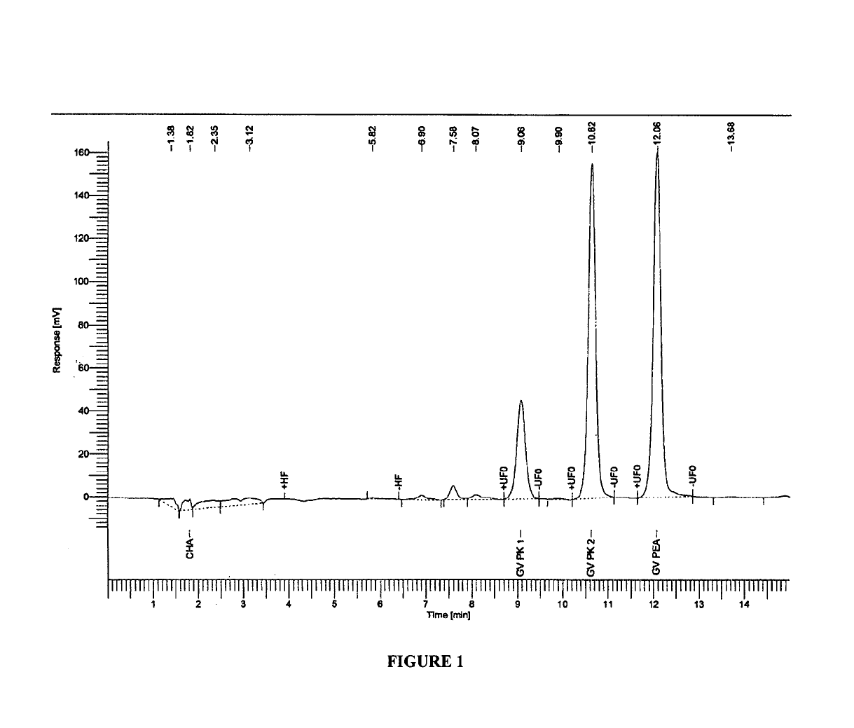

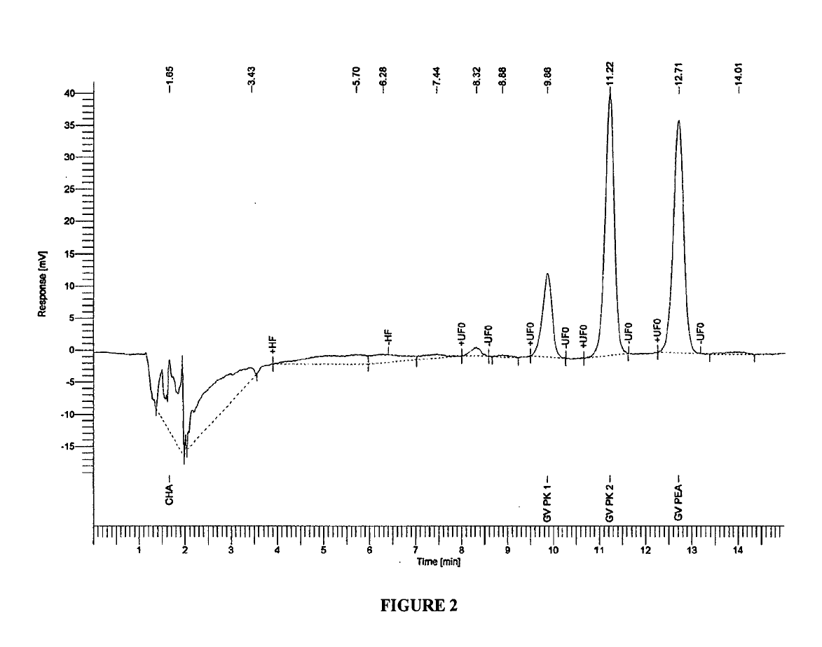

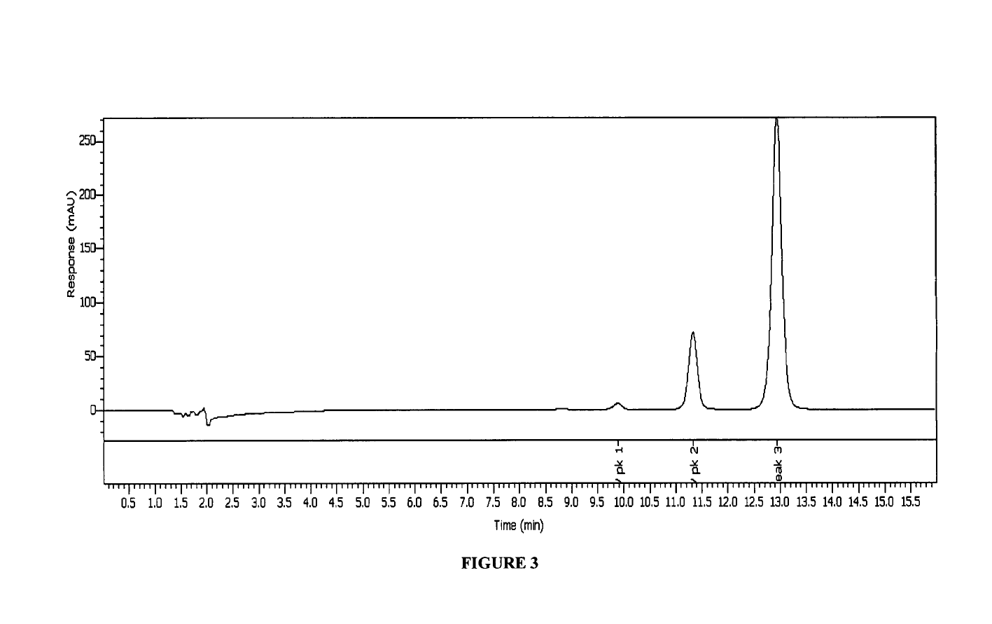

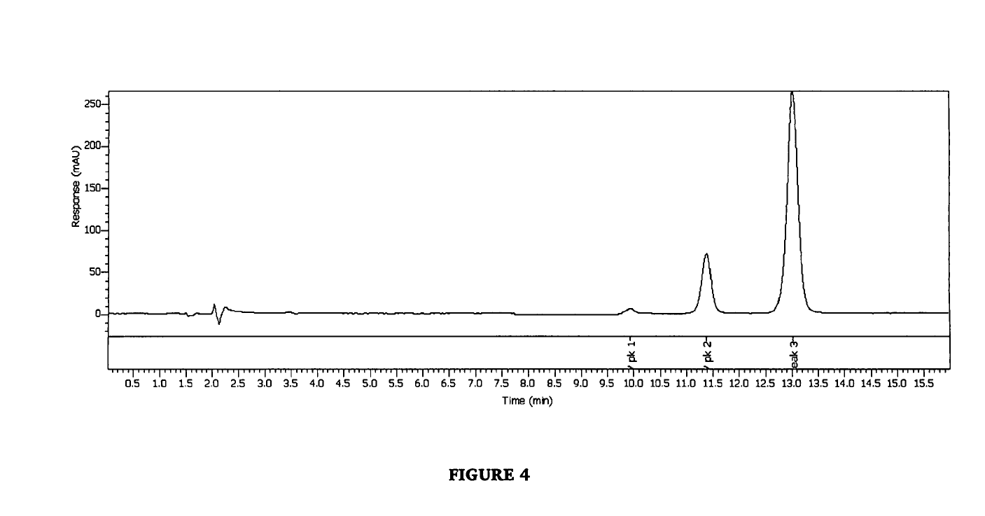

The gentian violet content of the compounded resin and extruded tube is shown in Table 1. Table 2 shows a comparison of the relative peak areas of gentian violet peaks of standard vs. sample. Percent area is based only on the total area of the three peaks, and not any other peaks shown in the chromatograms, which are shown in FIG. 1 and FIG. 2 for standard and sample (Sciencelab), respectively. Table 3 shows a comparison of the relative peak areas of gentian violet peaks of standard vs. sample (from Aldrich). FIG. 3 and FIG. 4 show the chromatographs of standard (Aldrich) and tube sample, respectively.

TABLE 1
GV content of compounded resin and extruded tube.

| | Theoretical | Measured GV loading |
|---|---|---|
| Sample | loading (% w/w) | (% w/w) via HPLC |
| Compounded | 0.5 | 0.67% |
| Resin | | |
| Tube | 2.0 | 2.2% |

TABLE 2
Relative % Areas of each peak, Standard vs Compounded sample.

| | |
|---|---|
| Standard | Sample |

| | | Relative | | Relative |
|---|---|---|---|---|
| Peak # | Area (uV-s) | % area | Area (uV-s) | % area |
| Peak 1 | 640917.67 | 14.75% | 191846.97 | 14.22% |
| Peak 2 | 1789022.3 | 41.17% | 583737.5 | 43.28% |
| Peak 3 | 1915778.8 | 44.08% | 573305.43 | 42.50% |
| Total area | 4345718.8 | | 1348889.9 | |

TABLE 3
Relative % Peak Area, Standard vs tube sample.

| | % Relative Peak area | | |
|---|---|---|---|
| | Peak 1 | Peak 2 | Peak 3 |
| Tube sample | 1.51 | 18.27 | 80.21 |
| Standard | 1.93 | 19.47 | 78.59 |

### Example III

### Temperature Effect on Degradation of Extruded Gentian Violet. Gentian Violet in Tecothane® Extrusions w/30% Filler

Extruded gentian violet was found to degrade at a processing temperature of 217° C. (starting at about 209° C.); however, processing under about 207° C. does not degrade gentian violet. One of the surprising findings about the temperature effects is that certain classes of polyurethanes are processable below the degradation temperature and other classes are not.

Compounding Conditions for stable gentian violet: Barrel Temperatures:

Zone 1-5=170° C.

Zone 6=175° C.,

Melt Temp=194° C.,

Screw Speed=100 RPM,

Pressure=˜180 bar.

Compounding Temperature for degraded gentian violet:

Zone 1-6=217° C.

Compounding was performed with Sigma Aldrich ACS grade gentian violet, lot 0161(3691. HPLC analysis was performed by cutting and measuring 1 cm segments of each formulation (n=3) using the method described in Examples I and II.

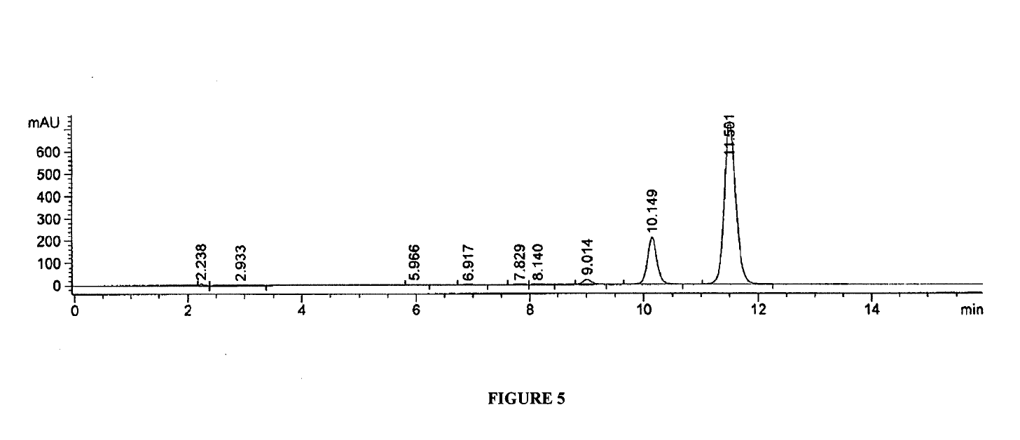

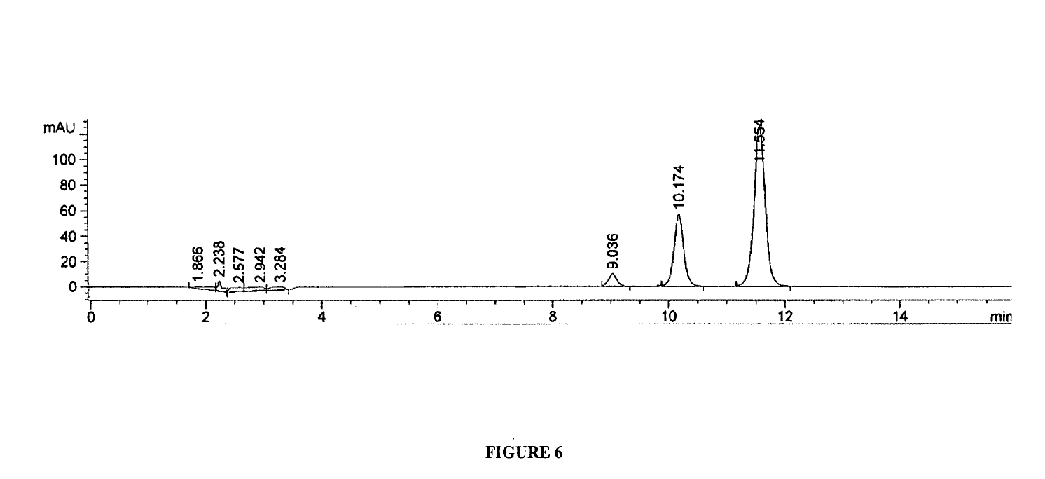

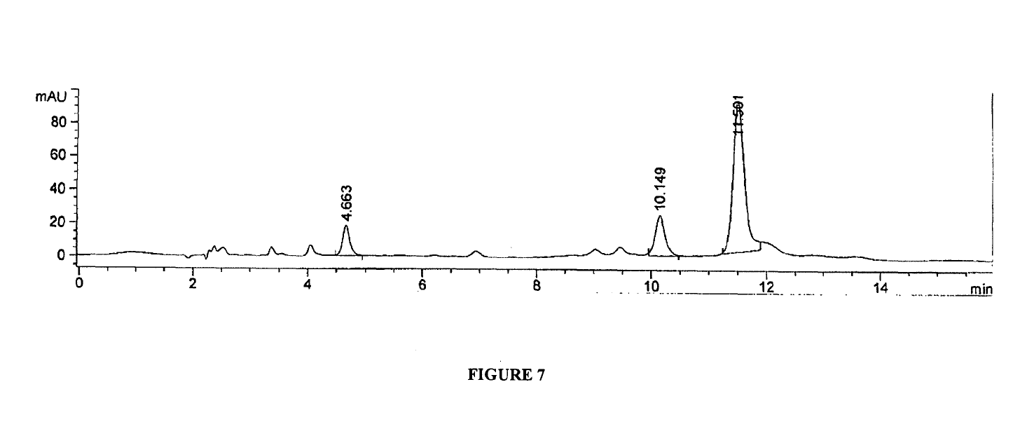

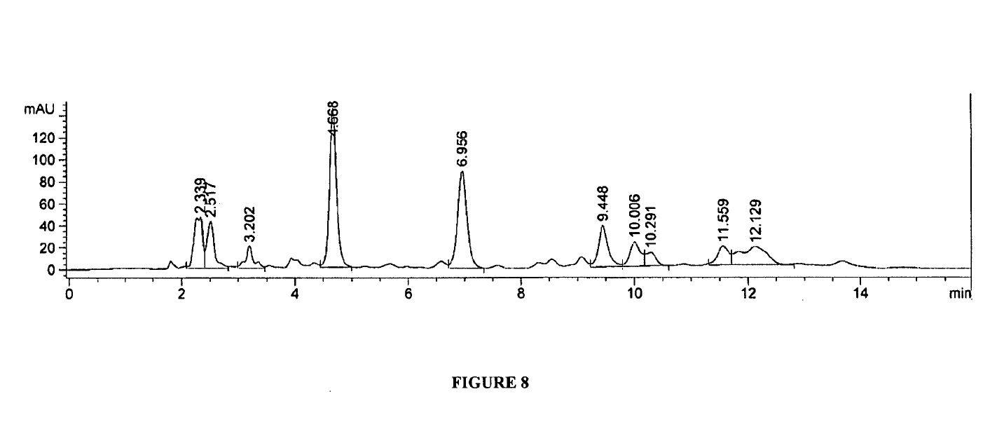

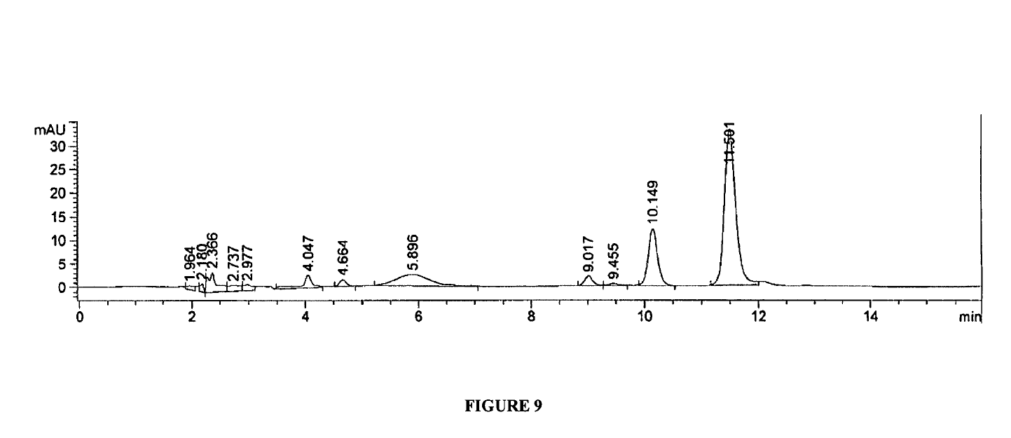

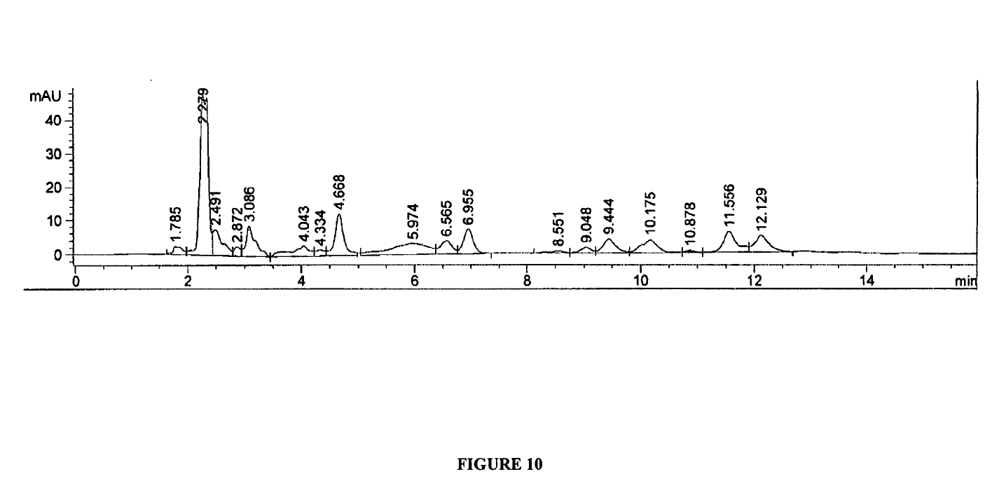

The chromatograms in FIGS. 5-10 show the stable gentian violet followed by the degraded gentian violet, at 588 nm, 253 nm, and 280 nm. The degradation peaks from the 217° C. sample are evident as the new peaks at 253 and 280 nm wavelengths. The chromatogram of the low processing temperature was identical to that of the Gentian Violet raw material.

### Example IV

### Temperature Effect (196° C.-219° C.) on Degradation of Extruded Gentian Violet. Compounded Carbothane® resin with 0.5% GV

5 g of gentian violet (Sigma-Aldrich, St. Louis, Mo.) was dissolved in 250 ml 99% ethanol (Sigma-Aldrich, St. Louis, Mo.). 1000 g of Carbothane®-3585A-B20 (Noveon, Cleveland, Ohio) resin was added in to the gentian violet/ethanol solution. The ethanol solvent was evaporated off in a chemical fume hood overnight at ambient conditions. The gentian violet coated pellets were then dried at 50° C. and 30 inches Hg for 24 hrs prior to compounding.

The dried gentian violet coated resins were starve-fed into a 18 mm Leistritz intermeshing twin screw extruder (Somerville, N.J.) from a K-iron feeder (Pitman, N.J.) at a rate of 2.5 kg/hr. The heating profile for each run was varied. The melt temperatures from each run are recorded in Table 4.

HPLC analysis was performed by selecting several pellets of each formulation (n=3) using the method described in Examples I and II.

GV content is essentially unchanged up to a processing temperature of 206° C. The onset of degradation appears at 209° C. and becomes worse at 219° C.

TABLE 4
GV Contents from compounded Carbothane ® at moderate temperatures.

| | Melt | GV measured from | GV measured from |
|---|---|---|---|
| | Temp | unprocessed resin | compounded resin (% w/w) |
| Sample # | (° C.) | (% w/w) | via HPLC |
| GV control | | 0.50 | |
| 3 | 196 | | 0.49 |
| 4 | 206 | | 0.51 |
| 5 | 209 | | 0.43 |
| 6 | 219 | | 0.40 |

### Example V

### Temperature Effect on Degradation of Extruded Gentian Violet-High Temperatures. Compounded Carbothane® resin with 0.5% GV

The sample preparation for loading GV on resin pellets is similar to EXAMPLE IV. Gentian violet was purchased from (Sigma-Aldrich, St. Louis, Mo.) and Carbothane®-3585A-B20 (Noveon, Cleveland, Ohio) was used. The heating profile was varied for each run. The melt temperatures are showed in Table 5.

There is significant degradation of the GV above 220° C. as evidenced by the dramatic drop in extractable GV content.

TABLE 5
GV contents from compounded Carbothane ® at high temperatures.

| | | GV measured from | GV measured from |
|---|---|---|---|
| | Melt Temp | unprocessed resin | compounded resin (% |
| Sample # | (° C.) | (% w/w) | w/w) via HPLC |
| GV control | | 0.45 | |
| 7 | 188 | | 0.42 |
| 8 | 221 | | 0.15 |
| 9 | 230 | | 0..13 |
| 10 | 235 | | 0.11 |
| 11 | 241 | | 0.09 |
| 12 | 246 | | 0.06 |

### Example VI

### Marking Implantation Site of Transcutaenous Catheter

This Example uses pig skin to simulate staining that would be apparent on a subject's skin after catheter implantation due to leaching of gentian violet from the catheter surface into surrounding tissue.

### Materials/Methods

### Sample Preparation

Catheter segments were either coated or impregnated. Coating was accomplished by dipping a catheter segment into a solution containing dye and a coating polymer (e.g., a polyurethane). Impregnation was accomplished by soaking in swelling solvent in which the dye was dissolved. The swelling solvent expanded the mesh of the polymer matrix and allowed the dye to diffuse into the wall of the catheter. Different concentrations of dye in the coating or swelling solutions were used to create segments with a range of dye loadings. The solvent was then evaporated off. The catheter shrunk back to approximately its original dimensions but the dye was entrapped within the polymer matrix.

Dip Coated Samples: Coating solution was prepared using:

258.5 g THF (JT Baker Lot C41832),

91.3 g Methanol (Fisher Lot 067887),

17.215 g 60D Lot CD5DRE024.

One stock solution of Gentian Violet (Science Lab) was prepared by adding 0.21 g to 150 mL coating solution (0.14%). Another stock solution of chlorhexidine diacetate (CHA) (Lot RZ3026851) was prepared by adding 3.75 g to 150 mL coating solution (2.5%). Formulations 1-9 were prepared according to Table 6.

TABLE 6
Formulations for coating solutions.

| | | | | | Ratio |
|---|---|---|---|---|---|
| Form# | GV (g) | CHA (g) | % GV | % CHA | CHA/GV |
| 1 | 0.03 | 0.03 | 0.14 | 0.16 | 1.06 |
| 2 | 0.03 | 0.14 | 0.14 | 0.71 | 4.72 |
| 3 | 0.03 | 0.34 | 0.14 | 1.70 | 11.19 |
| 4 | 0.03 | 0.56 | 0.14 | 2.79 | 19.96 |
| 5 | 0.03 | 0.50 | 0.12 | 2.51 | 20.00 |
| 6 | 0.05 | 0.50 | 0.25 | 2.50 | 9.89 |
| 7 | 0.10 | 0.50 | 0.49 | 2.49 | 4.99 |
| 8 | 0.20 | 0.50 | 0.97 | 2.49 | 2.50 |
| 9 | 0.25 | 0.50 | 1.21 | 2.47 | 2.00 |

GV = gentian violet;
CHA = chlorhexidine diacetate.

Impregnated Samples: Stock impregnation solution was prepared at the following ratio:

85% Methyl ethyl ketone (Fisher lot 067353),

10% Deionized Water,

5% Acetone (Fisher Lot 063123).

A stock solution of gentian violet for Set 1 was prepared by adding 0.34996 g into 250 mL (0.14%) impregnation solvent. A stock solution of chlorhexidine base (CHX) was prepared for Set 2 by adding 8.75 g in 350 mL (2.5%) impregnation solvent. A stock solution of gentian violet for Set 3 was prepared by adding 1.000 g in 200 mL stock solution. Formulations for Set 3 were prepared by serial dilutions of the stock. Formulations for Sets 1-3 were prepared according to Table 7.

TABLE 7
Formulations for impregnation solutions.

| | | | | | Ratio |
|---|---|---|---|---|---|
| Form# | GV (g) | CHX (g) | % GV | % CHX | CHX/GV |
| 10 | | 0.84 | 0.14 | 0.14 | 1.00 |
| 11 | | 0.42 | 0.14 | 0.70 | 5.00 |
| 12 | | 1.01 | 0.14 | 1.68 | 12.00 |
| 13 | | 1.68 | 0.14 | 2.80 | 20.00 |
| 14 | 0.08 | | 0.13 | 2.50 | 20.00 |
| 15 | 0.15 | | 0.25 | 2.50 | 10.00 |
| 16 | 0.30 | | 0.50 | 2.50 | 5.00 |
| 17 | 0.60 | | 1.00 | 2.50 | 2.50 |
| 18 | 0.75 | | 1.25 | 2.50 | 2.00 |
| 19 | | | 0.50 | | |
| 20 | | | 0.10 | | |
| 21 | | | 0.05 | | |
| 22 | | | 0.01 | | |

GV = gentian violet;
CHX = chlorhexidine base.

Samples were dried in an oven under vacuum at 50° C. for 24 hrs, rinsed with methanol, and dried again under vacuum overnight to complete preparation.

Drug Content Analysis: Drug content analysis was performed by cutting and measuring 1 cm catheter segments of each formulation (n=3) and digesting in THF (10 mL or 20 mL) in 50 mL centrifuge tubes. Samples were allowed to sit for 45 minutes, and then vortexed for 5 minutes, or until none of the polymer was stuck to the bottom of the tubes. An equal amount of DI water was then added (10 or 20 mL) and the samples were again vortexed for 5 minutes, and then centrifuged for 10 minutes. A portion of each sample was then added to an HPLC vial and capped prior to HPLC analysis.

Analysis was performed on an Agilent 1200 Series LC using an Agilent Eclipse XDB-CN 5μ4.6×150 mm column with the corresponding guard column. A gradient program was run using two solvent reservoirs:

MP A: 100% DI Water/0.2% Trifluoroacetic Acid,

MP B: 100% Acetonitrile/0.2% Trifluoroacetic Acid. Program: 0-5 min 35% B, 5-6 min 35-40% B, 6-12 min 40% B, 12-16 min 35% B. Wavelength A=280 nm; Wavelength B=588 nm. Concentrations were calculated based on a six point standard curve.

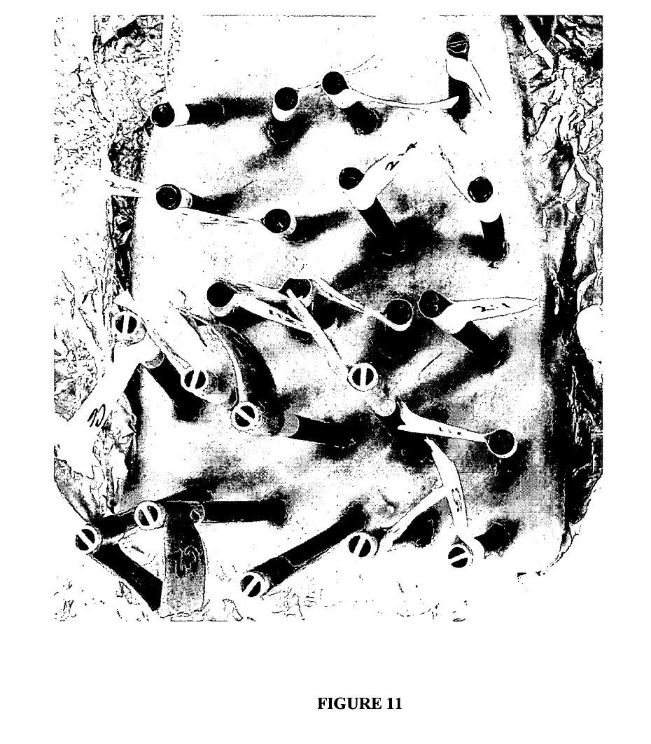

Pig Skin Preparation: A fresh section of swine dermis was obtained from a local abattoir and refrigerated prior to use. A sterile scalpel was utilized to cut 22 small holes into the pig skin. The holes were cut slightly smaller that the catheter segment diameter to assure a snug fit. Twenty-two different catheter treatments representing different ratios and concentrations of gentian violet and chlorhexidine were evaluated. Impregnated samples used chlorhexidine base and coated samples used chlorhexidine diacetate. The catheter segments were labeled and inserted into the pig skin, which was then sealed in a plastic container and incubated 24 hours at 37° C. The pig skin staining setup is shown in FIG. 11.

### Results

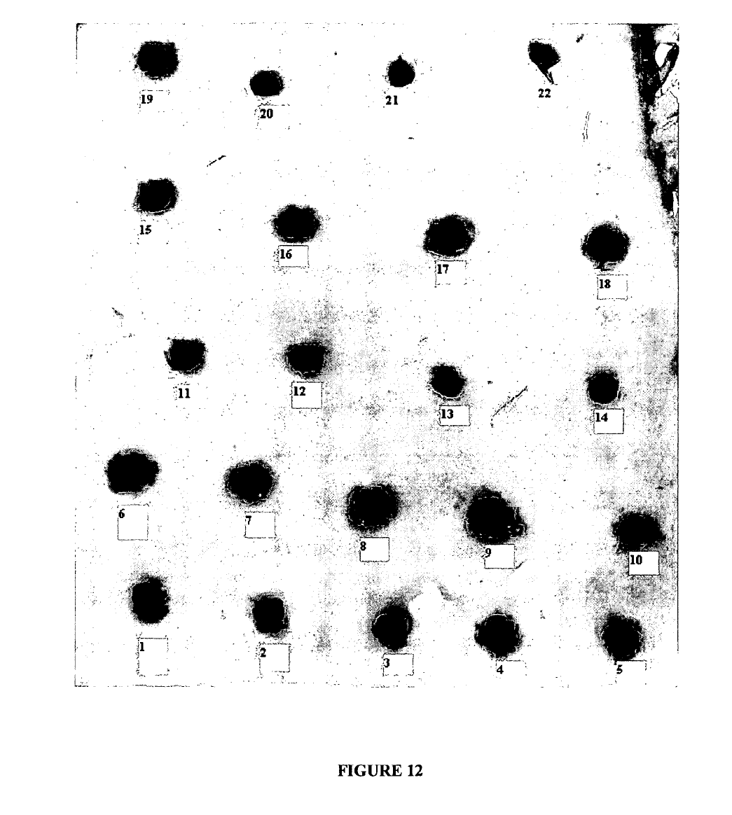

After 24 hours of incubation the catheter segments were pulled out and the surrounding skin was examined for staining to delineate where the catheter had resided (FIG. 12). The number code for the different catheter segments in Tables 6-9 is attached to the photograph of the pig skin in FIG. 12 to allow correlation between the degree of staining and the treatment condition. Sample analysis is shown in Tables 8 and 9. Numbers are averages of 3 replicates, and relative standard deviations (RSD) are shown. Over half of the RSDs are below 5%, and over 90% are lower than 10%, showing that the sample analysis and preparation was consistent. All twenty-two formulations were effective in marking the surface of the skin and/or the tunnel track of the catheter, as shown in FIG. 12.

TABLE 8
Dip coated sample results.

| | CHA | | GV | |
|---|---|---|---|---|
| Form # | μg/cm | rsd | μg/cm | rsd |
| 1 | 149.23 | 3.88 | 72.60 | 4.56 |
| 2 | 545.76 | 4.45 | 84.76 | 4.99 |
| 3 | 1092.74 | 14.07 | 79.42 | 13.00 |
| 4 | 1693.10 | 6.96 | 73.55 | 6.18 |
| 5 | 1858.10 | 14.59 | 112.28 | 13.01 |
| 6 | 2282.08 | 7.43 | 211.60 | 7.96 |
| 7 | 2028.41 | 4.62 | 432.38 | 4.90 |
| 8 | 1898.43 | 5.19 | 862.34 | 5.42 |
| 9 | 1433.97 | 2.57 | 662.80 | 2.83 |

All numbers are averages of 3 replicates.

TABLE 9
Impregnated sample results.

| | | CHX | average | | GV | average | |
|---|---|---|---|---|---|---|---|
| Form # | Description | %/polymer | μg/cm | rsd | %/polymer | μg/cm | rsd |
| 10 | 0.14% GV/0.14% CHX | 0.16 | 245.96 | 1.81 | 0.21 | 329.24 | 2.89 |
| 11 | 0.14% GV/0.7% CHX | 0.81 | 1263.95 | 3.01 | 0.13 | 202.34 | 2.98 |
| 12 | 0.14% GV/1.68% CHX | 2.00 | 3067.07 | 2.89 | 0.09 | 135.06 | 5.72 |
| 13 | 0.14% GV/2.8% CHX | 3.48 | 5037.70 | 6.16 | 0.07 | 95.25 | 9.38 |
| 14 | 0.125% GV/2.5% CHX | 2.07 | 3272.41 | 4.05 | 0.05 | 80.07 | 5.24 |
| 15 | 0.25% GV/2.5% CHX | 2.05 | 3193.27 | 1.76 | 0.11 | 174.62 | 3.01 |
| 16 | 0.5% GV/2.5% CHX | 1.94 | 2948.60 | 1.25 | 0.24 | 369.23 | 1.01 |
| 17 | 1% GV/2.5% CHX | 1.76 | 2714.77 | 1.51 | 0.51 | 769.01 | 3.26 |
| 18 | 1.25% GV/2.5% CHX | 1.58 | 2489.06 | 2.14 | 0.58 | 950.97 | 5.33 |
| 19 | 0.5% GV | | | | 0.51 | 803.31 | 2.15 |
| 20 | 0.1% GV | | | | 0.10 | 153.21 | 0.75 |
| 21 | 0.05% GV | | | | 0.05 | 81.22 | 1.53 |
| 22 | 0.01% GV | | | | 0.02 | 25.96 | 2.45 |

All numbers are averages of 3 replicates.

### Example VII

### Dermal Decolorization of Gentian Violet Using Hydroxyl Radicals

### Overview

As described in the present application, gentian violet (GV) can leech out from a treated catheter, leaving a dermal stain around the site of insertion of the catheter. A means of removing this stain, after extraction of the catheter, can provide a cosmetic benefit. Decolorization of dye effluents has acquired increasing attention within the textile industry since dye wastewater pollutants are sources of environmental pollution (Roxon et al. 1967). Physical and chemical decolorization of GV has been investigated (Saquib and Muneer 2003). It has been reported that decolorization of GV was achieved by oxidation.

In the present example, the use of hydrogen peroxide (H₂O₂) as the oxidizing species was investigated but the method of activating it was altered. The decolorization of gentian violet by H₂O₂ was investigated by three different activation techniques: photolytic split of H₂O₂ (by UV light), decomposition of H₂O₂ by non-ionic silver (Ag) nanoparticles, and decomposition of H₂O₂ by ascorbic acid (Vitamin-C). It was determined that certain methods of activation are able to convert the GV to a colorless (oxidized) form.

### Materials/Methods

The following material were used: Crystal Violet ACS (lot # 036K0709, Sigma-Aldridge) (Gentian violet and Crystal violet are interchangeable names of the same compound), Hydrogen Peroxide (30% ACS lot# 060350, Fisher Scientific), Nano-particle Silver, Ascorbic Acid, and TRIS (Hydroxymethyl Aminomethane) (lot #062202, Fisher BioReagents).

The following three protocols were evaluated:

Protocol 1. Photo-oxidation of GV with H₂O₂
1.1. 0.15 g GV (0.1% w/v) was dissolved in 150 ml 3% H₂O₂ The solution was exposed to UV light (Bio-lab hood) for 2 hours. 5 ml of solution was extracted out, in increments of 5 mins.
1.2. 10 μl of 0.1% GV in DI water was spiked onto a plated agar plate. After staining of the agar plate (evaporation of most of the water from the spiked drop), H₂O₂ was pipetted onto the stain in increments of 50 μl every 5 mins for 2 hours, under the UV light.
   Protocol 2. Oxidation of GV by activation of H₂O₂ with Ag
2.1. 10.6 mg of nano-particle Silver was mixed into 40 ml 3% H₂O₂ with constant stirring. 0.1% GV in water was added at various increments (100-1 ml) until purple color persisted. Molar concentration ratios (GV: Ag) were calculated to determine the molar ratio where GV is no longer oxidized.
2.2 10 μl of 0.1% GV in DI water was spiked onto a plated agar plate. After staining of the agar plate (evaporation of most of the water from the spiked drop), 0.009 g of Ag was applied to the surface of the stain and 1000 of H₂O₂ was pipette onto the Ag.
   Protocol 3. Oxidation of GV by activation of H₂O₂ with Vitamin C
1% Vitamin C (w/v) was dissolved in 100 ml 3% H₂O₂ 7 g of TRIS buffer was also dissolved in the H₂O₂. 0.1% GV in water was added at increments of 10 μl until the purple color persisted.

### Results and Discussion

Protocol 1. No color change was observed in all test samples treated with UV light. Solution and agar stain remained purple after numerous hours under UV light. Since it has been reported that H₂O₂ splits photolytically to produce OH (Peyton and Glaze 1988), the intensity of the UV light used here might not have been adequate to generate these radicals.

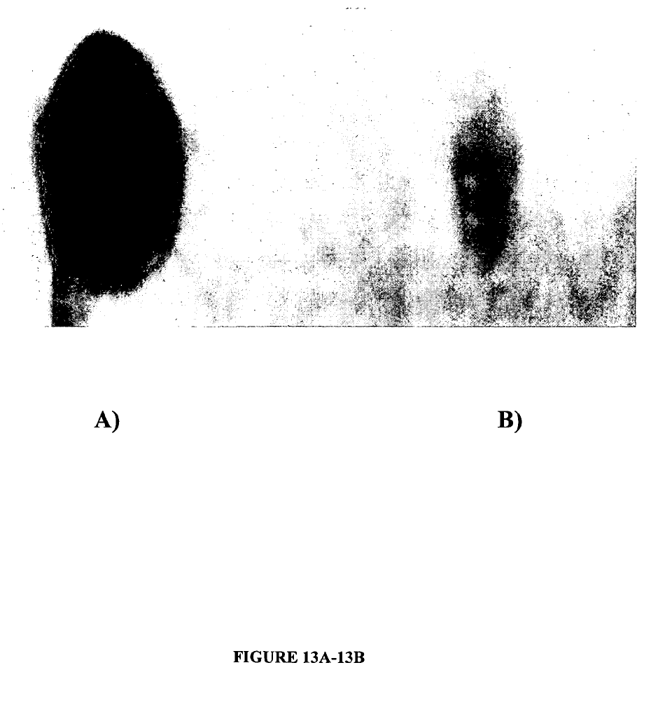

Protocol 2. As the 0.1% GV solution was pipetted in the Ag+H₂O₂ solution, the purple color was observed to fade to a faint yellow color. This faint yellow color became darker as more GV was added, until the purple color remained permanently. The molar ratio of GV:Ag at this point was calculated to be 1:1. Similarly, the purple stain on the agar was observed to turn to a faint yellow color after 10 mins (FIG. 13B).

Protocol 3. The addition of Vitamin C to H₂O₂ was observed to reduce the pH of the solution. IRIS buffer was added to neutralize the acidic solution to a pH range of 6-7. With the addition of the first 10 μl increment of 0.1% GV, the solution turned and remained purple.

In all test techniques, only with the use of non-ionic silver was hydrogen peroxide able to neutralize the purple color from GV (from purple to faint yellow). Neither vitamin C nor UV light was a strong enough activating agent, at the concentrations tested here.

### REFERENCES

Peyton G R, Glaze W H. Destruction of pollutant in water with ozone in contamination with ultraviolet radiation. Environ Sci Technol 1988; 22:761-7.
Roxon J J, Ryan A J, Wright SE. Reduction of water-soluble azo dyes by intestinal bacteria. Food Cosmet Toxicol 1967; 5: 367-9.
Saquib, M. Muneer, M. TiO₂-mediated photocatalytic degradation of a triphenylmethane dye (gentian violet), in aqueous suspensions. Dyes and Pigments 56 (2003) 37-49.
PCT International Publication No. WO 03/093338 A2, published Nov. 13, 2003, Biopsy Sciences, LLC, Biodegradable polymer for marking tissue and sealant tracts.
PCT International Publication No. WO 2006/022786 A1, published Mar. 2, 2006, Mullen, Tissue marking devices and systems.
U.S. Patent Application Publication No. US 2003/0078242 A1, published Apr. 24, 2003, Raad et al. Novel antiseptic derivatives with broad spectrum antimicrobial activity for the impregnation of surfaces.
U.S. Patent Application Publication No. US 2005/0131356 A1, published Jun. 16, 2005, Ash et al. Medical devices exhibiting antibacterial properties.
U.S. Patent Application Publication No. US 2005/0197634 A1, published Sep. 8, 2005, Raad et al. Methods for coating an impregnating medical devices with antiseptic compositions.
U.S. Patent Application Publication No. 2006/0200024, published Sep. 7, 2006, Knapp, System and method for marking body cavities.
U.S. Pat. No. 5,147,307, issued Sep. 15, 1992, Gluck, Anatomical marker device and method.
U.S. Pat. No. 5,192,270, issued Mar. 9, 1993, Carswell, Jr., Hypodermic syringe and a method for marking injections.
U.S. Pat. No. 5,607,417, issued Mar. 4, 1997, Batich et al., Compositions and devices for controlled release of active ingredients.
U.S. Pat. No. 5,709,672, issued Jan. 20, 1998, Illner, Silastic and polymer-based catheters with improved antimicrobial/antifungal properties.
U.S. Pat. No. 5,713,858, issued Feb. 3, 1998, Heruth et al., Permanently implantable guiding catheter.
U.S. Pat. No. 6,228,055, issued May 8, 2001, Foerster et al., Devices for marking and defining particular locations in body tissue.
U.S. Pat. No. 6,361,786, issued Mar. 26, 2002, Shanbrom, Microbicide treated polymeric materials.
U.S. Pat. No. 6,551,346, issued Apr. 22, 2003, Crossley et al., Method and apparatus to prevent infections.
U.S. Pat. No. 6,617,142, issued Sep. 9, 2003, Keogh et al., Method for attachment of biomolecules to medical device surfaces.
U.S. Pat. No. 6,780,179, issued Aug. 24, 2004, Lee et al. Methods and systems for in situ tissue marking and orientation stabilization.

1. A method of marking an insertion site and/or an exit site of a transcutaneous device, and/or the track of the device through the subject, the method comprising inserting a transcutaneous device through the skin of the subject, where the transcutaneous device provides a cue in an amount effective to mark the insertion site, the exit site and/or the track of the transcutaneous device.

2-6. (canceled)

7. The method of claim 1, wherein the transcutaneous device provides a cue on the surface of the skin to mark the insertion site and/or the exit site.

8. The method of claim 1, wherein the transcutaneous device provides a cue on the surface of the skin to delineate the circumference of the portion of the transcutaneous device that is inserted through the skin.

9. The method of claim 1, wherein the transcutaneous device provides a cue to mark all or part of the track of the device between the insertion site and the exit site.

10-16. (canceled)

17. The method of claim 1, wherein the transcutaneous device is coated with the cue.

18. The method of claim 1, wherein the transcutaneous device is impregnated with the cue.

19-20. (canceled)

21. The method of claim 1, wherein the cue is gentian violet.

22. The method of claim 21, wherein gentian violet is present in a portion of the transcutaenous device that passes through the skin in a concentration greater than 1% by weight of the portion of the device that passes through the skin.

23-25. (canceled)

26. The method of claim 1, wherein the transcutaneous device is selected from the group consisting of a suture, a staple, a trocar, a tube, a multilumen tube, multiple tubes, a fixation pin, a fixation clip, a wire, a cable and a catheter.

27. (canceled)

28. A transcutaneous device for marking an insertion site and/or an exit site of the device through the skin of a subject, and/or the track of the device through the subject, where the transcutaneous device comprises a cue in an amount effective to mark the insertion site, the exit site and/or the track of the transcutaneous device.

29-40. (canceled)

41. The transcutaneous device of claim 28, wherein the transcutaneous device is coated with the cue.

42. The transcutaneous device of claim 28, wherein the transcutaneous device is impregnated with the cue.

43-44. (canceled)

45. The transcutaneous device of claim 28, wherein the cue is gentian violet.

46. The transcutaneous device of claim 45, wherein gentian violet is present in a portion of the transcutaenous device that passes through the skin in a concentration greater than 1% by weight of the portion of the device that passes through the skin.

47-49. (canceled)

50. The transcutaneous device of claim 28, wherein the transcutaneous device is selected from the group consisting of a suture, a staple, a trocar, a tube, a multilumen tube, multiple tubes, a fixation pin, a fixation clip, a wire, a cable and a catheter.

51. (canceled)

52. A method of decoloring a dye stain on the surface of the dermis and/or along the track of a transcutaneous device, the method comprising applying peroxide to the stain and treating the peroxide with non-ionized metal particles.

53. The method of claim 52, wherein the non-ionized metal comprises non-ionized silver, gold, platinum, copper, iron or zinc.

54-61. (canceled)

62. A kit for removing a dye stain on the surface of the dermis and/or along the track of a transcutaneous device, the kit comprising peroxide and non-ionized metal particles.

63. A transcutaneous device kit for marking an insertion site and/or an exit site of the transcutaenous device on the dermis and/or marking the track of the transcutaneous device, and for decoloring a dye stain on the surface of the dermis and/or along the track of the transcutaneous device, the kit comprising:
a) a transcutaneous device that provides a visible dye mark on the surface of the skin to mark the insertion site and/or the exit site and/or a dye mark along the track of the transcutaneous device, and
b) non-ionized metal particles.

64. (canceled)

65. The kit of claim 62, wherein the non-ionized metal comprises non-ionized silver, gold, platinum, copper, iron or zinc.

66-72. (canceled)
